(19) United States
(12) Patent Application Publication
(10) Pub. No.: US 2012/0065436 A1
(43) Pub. Date: Mar. 15, 2012

(54) PROCESS FOR PREPARING FLUORINE-CONTAINING PROPANE

(75) Inventors: Daisuke Karube, Osaka (JP); Tsuneo Yamashita, Osaka (JP); Akinarri Sugiyama, Osaka (JP); Takashi Shibanuma, Osaka (JP)

(73) Assignee: DAIKIN INDUSTRIES, LTD., Osaka (JP)

(21) Appl. No.: 13/319,588

(22) PCT Filed: May 11, 2010
(86) PCT No.: PCT/JP2010/058247

§ 371 Date: Nov. 9, 2011

Related U.S. Application Data
(60) Provisional application No. 61/213,154, filed on May 12, 2009.

(51) Int. Cl. C07C 17/281

(52) U.S. Cl. 570/171

(57) ABSTRACT

The present invention provides a process for preparing a fluorine-containing propane represented by the formula: CF₂XCF₂CH₃ wherein X is F or Cl, the process including reacting tetrafluoroethylene and methyl chloride in the presence of an antimony halide represented by the formula: SbFₓC₅₋ₓ wherein x is a value of 0 to 5. According to the present invention, the fluorine-containing propane represented by the formula: CF₂XCF₂CH₃ wherein X is F or Cl, which is useful as a starting material of 2,3,3,3-tetrafluoropropene (1234yf), can be obtained by a simple process, using relatively inexpensive starting materials.

### TECHNICAL FIELD

The present invention relates to a process for preparing a fluorine-containing propane represented by the formula: CF₂XCF₂CH₃ wherein X is F or Cl.

### BACKGROUND ART

A known method for producing 2,3,3,3-tetrafluoropropene (1234yf) represented by the formula: CF₃CF=CH₂ comprises subjecting hydrofluorocarbon represented by Formula (1): CF₂X₁CF₂CH₃ wherein X₁ represents F, Cl, Br, or I to dehydrohalogenation reaction, thereby obtaining 1234yf. However, since the hydrofluorocarbon starting material represented by Formula (1) is obtained through several production steps using tetrafluoroethylene and halogenated methane as starting materials, this method requires a prolonged reaction process, and is thus economically disadvantageous.

As a method for producing hydrofluorocarbon represented by Formula (1) above, Patent Literatures 1 and 2 listed below disclose that CF₃CF₂CH₃ or CF₂ClCF₂CH₃ can be prepared in a single step by reacting tetrafluoroethylene or chlorotrifluoroethylene with methyl fluoride in the presence of a Lewis acid catalyst. However, this method has a disadvantage in that methyl fluoride is expensive.

### Citation List

Patent Literature

PTL 1 U.S. Pat. No. 6,184,426B1

PTL 2 US2006-258891A1

### SUMMARY OF INVENTION

### Technical Problem

The present invention is made in view of the problems of the prior art described above. A main object of the present invention is to provide a simple process for preparing a fluorine-containing propane represented by the formula: CF₂XCF₂CH₃ wherein X is F or Cl, which is useful as a starting material for producing 2,3,3,3-tetrafluoropropene (1234yf), using relatively inexpensive starting materials.

### Solution to Problem

The present inventors conducted extensive research to achieve the above object. As a result, they found that the target fluorine-containing propane represented by the formula: CF₂XCF₂CH₃ wherein X is F or Cl can be efficiently obtained at low cost by a simple process comprising a single step of reacting inexpensive starting materials, i.e., tetrafluoroethylene and methyl chloride, in the presence of an antimony halide represented by a specific chemical formula. The present invention was thus accomplished.

Specifically, the present invention provides the following processes for preparing a fluorine-containing propane.
1. A process for preparing a fluorine-containing propane represented by the formula: CF₂XCF₂CH₃ wherein X is F or Cl, the process comprising reacting tetrafluoroethylene and methyl chloride in the presence of an antimony halide represented by the formula: SbFₓCl₅₋ₓ wherein x is a value of 0 to 5.
2. The process according to Item 1, wherein the antimony halide is antimony pentafluoride.
3. The process according to Item 2, wherein the antimony pentafluoride is obtained by bringing an antimony halide represented by the formula: SbFₓCl₅₋ₓ, wherein x is a value of not less than 0 and less than 5, into contact with a fluorinating agent to convert the antimony halide into the antimony pentafluoride.
4. The process according to Item 1, wherein the antimony halide is used in an amount of 0.01 to 3 mol per mol of tetrafluoroethylene.
5. The process for preparing a fluorine-containing propane according to Item 1, wherein the antimony halide is prepared by bringing the antimony halide, which has been used in the production of fluorine-containing propane by the process of Item 1, into contact with an oxidizing agent and/or a fluorine-containing compound to thereby reactivate the antimony halide.
6. The process according to Item 5, wherein the oxidizing agent is chlorine, and the fluorine-containing compound is hydrogen fluoride.

In the production process of the present invention, tetrafluoroethylene and methyl chloride used as starting materials are reacted in the presence of an antimony halide represented by the formula: SbFₓCl₅₋ₓ wherein x is a value of 0 to 5.

Antimony halides used as catalysts are not particularly limited as long as they are represented by the formula: SbFₓCl₅₋ₓ wherein x is a value of 0 to 5. In particular, antimony halides represented by the above formula wherein x is 5, namely, antimony pentafluorides represented by the formula: SbF₅, are preferable in view of reactivity. However, in general, as the value x in the formula: SbFₓCl₅₋ₓ becomes larger, the corrosivity increases; therefore, an antimony halide having an appropriate value x is used in accordance with production equipment to be used.

Although there is no particular limitation on the process for reacting tetrafluoroethylene and methyl chloride in the presence of an antimony halide, the reaction is generally performed in the liquid phase. For example, the reaction can proceed by adding gaseous or liquefied methyl chloride and gaseous tetrafluoroethylene to an antimony halide in a liquid state, or in a simultaneous liquid/solid state (i.e., a slurry state), to dissolve or disperse the methyl chloride and tetrafluoroethylene in the antimony halide.

In a specific embodiment of the reaction method, the reaction can be conducted by sequentially or simultaneously adding methyl chloride and tetrafluoroethylene to a reaction vessel in which an antimony halide has been supplied under a dry atmosphere. Methyl chloride and tetrafluoroethylene are added in no particular order. In this case, it is desirable that an antimony halide in a liquid or slurry state be stirred after the addition of the starting materials. Alternatively, in the case when methyl chloride is partially liquefied, it is desirable that a liquid mixture or slurry of antimony halide and liquefied methyl chloride be stirred after the addition of the starting materials.

The amount of methyl chloride is preferably about 0.1 to 10 mol, and more preferably about 0.5 to 2 mol per mol of tetrafluoroethylene. In particular, it is preferable that tetrafluoroethylene and methyl chloride be used in nearly equimolar amounts. When the molar amounts therebetween are significantly different, a needlessly large reaction vessel with respect to the amount of the resulting target compound is required, which is disadvantageous.

The amount of antimony halide is preferably about 0.01 to 3 mol per mol of tetrafluoroethylene. In particular, from an economic point of view, the antimony halide is preferably used in an equimolar amount or less to tetrafluoroethylene, i.e., about 0.01 to 1 mol per mol of tetrafluoroethylene.

In the production process of the present invention, a solvent may be added to increase the stirring efficiency and contact efficiency of the components, and to reduce the pressure during reaction by dissolving the gas components. The weight of solvent is about 1 to 100 times, preferably about 1 to 10 times as large as that of antimony halide supplied. When an excess amount of solvent is used, the antimony halide is excessively diluted, which may retard or stop the reaction. Further, an overly large amount of solvent requires an unnecessarily large reaction vessel, which is not preferable.

Solvents are not particularly limited as long as they do not react with starting materials, resulting products, and the like; are able to dissolve or disperse components to be used in the reaction; have little effect on the oxidation nature, halogen-abstracting properties, etc. of antimony halide; and have a boiling point that is not so lower than the reaction temperature. For example, fluorocarbon-based solvents can be used, preferably perfluoroalkanes of alkanes having about 4 to 8 carbon atoms, such as hexane.

The reaction temperature is not particularly limited. In general, the reaction temperature is preferably about room temperature to 200° C., more preferably about room temperature to 110°. An overly low reaction temperature retards the reaction, whereas an overly high reaction temperature causes a side reaction, and results in high equipment corrosivity. Thus, a reaction temperature outside the above range is not preferable.

The pressure during the reaction is not particularly limited. In general, about atmospheric pressure (0.1 MPa) to 1 MPa is preferable. A high pressure is advantageous because tetrafluoroethylene and methyl chloride have high contact efficiency, which accelerates the reaction speed. However, an overly high pressure is unfavorable, because the side reaction of tetrafluoroethylene easily occurs.

When antimony pentafluoride is used as an antimony halide, in place of directly supplying antimony pentafluoride into a reaction vessel, an antimony halide represented by the formula: SbFₓCl₅₋ₓ wherein x is less than 5, e.g., antimony pentachloride, can be supplied to the reaction vessel and then converted into antimony pentafluoride. In this case, after sealing the reaction equipment in which the antimony halide has been supplied into the reaction vessel, a fluorinating agent such as hydrogen fluoride can be added to the reaction equipment to react with the antimony halide. When antimony pentachloride is used as an antimony halide, the amount of hydrogen fluoride is about 5 to 1,000 mol, and preferably about 100 to 1,000 mol, per mol of antimony pentachloride. After hydrogen fluoride is added to the reaction vessel, the antimony halide can be converted into antimony pentafluoride by adjusting the temperature in the range of about room temperature to about 110° C. while stirring the antimony halide in the reaction vessel. In this case, the pressure of the system is increased due to the generation of hydrogen chloride. By removing the hydrogen chloride from the system using a pressure-adjusting valve, the conversion reaction can be accelerated.

In the production process of the present invention, the antimony halide used once in the reaction can be reused after the reaction is completed. For example, after completion of the reaction, the resulting products and volatile components such as starting material gas are removed from the reaction vessel and transferred to another vessel at a temperature less than a boiling point of the antimony halide, and then the methyl chloride and tetrafluoroethylene that are used in the next reaction are supplied into the reaction vessel to repeat the aforementioned operation; thereby, the antimony halide used in the preceding reaction can be reused to repeat the reaction.

In the process of the present invention, since the oxidation number and the fluorine-containing ratio of antimony halide are reduced as a result of repeated reaction, the activity of the antimony halide is decreased, which may gradually slow down the reaction speed. In this case, the decreased activity of the antimony halide can be restored by allowing the antimony halide to react with an oxidizing agent and/or a fluorine-containing compound (hereinafter, both referred to as reactivation agents). Usable oxidizing agents include compounds that are capable of converting an antimony halide into pentavalent antimony halide (e.g., chlorine); and usable fluorine-containing compounds include those that are capable of increasing the fluorine-containing ratio of antimony halide (e.g., hydrogen fluoride).

In the present invention, after completion of the reaction of tetrafluoroethylene and methyl chloride, reactivation treatment can be conducted in the reaction vessel, by removing volatile components such as starting material gas and resulting products other than the antimony halide from the reaction vessel, and adding the aforementioned reactivation agent to the reaction vessel. Alternatively, by adding the reactivation agent to the reaction system during reaction in a manner such that the reaction of tetrafluoroethylene and methyl chloride is not adversely affected, a decrease in activity of the antimony halide used in the reaction can be inhibited. It is also possible to take out the antimony halide having lowered activity from the reaction system after first completion of the reaction, and reactivate it using a reactivation agent.

The amount of reactivation agent varies depending on the degree of decrease in activity of the antimony halide, and the corrosion resistance of the reaction equipment. In general, the amount of reactivation agent is preferably about 0.1 to 50 mol, per mol of antimony halide supplied. Reactivation treatment is generally performed by stirring the reaction components at a temperature of about ordinary temperature to about 110° C., and a pressure of about ordinary pressure to about 2 MPa.

According to the process of the invention, a fluorine-containing propane represented by the formula: CF₂XCF₂CH₃ wherein X is F or Cl can be obtained. Specifically, two types of fluorine-containing propanes, i.e., a compound (HFC-245cb) represented by CF₂FCF₂CH₃, and a compound (HCFC-244cc) represented by CF₂ClCF₂CH₃, are mainly obtained. The production ratio of these two types of compounds can be controlled according to the reaction conditions, particularly the reaction time and reaction temperature. The production ratio can be also adjusted based on the value x of antimony halide used, i.e., the amount of fluorine. In general, the production ratio of HFC-245cb tends to increase as the reaction time is prolonged and the reaction temperature rises.

The fluorine-containing propane obtained by the process according to the present invention is highly useful as a starting material for producing 2,3,3,3-tetrafluoropropene (1234yf).

### Advantageous Effects of the Invention

According to the process of the present invention, a fluorine-containing propane represented by the formula: CF₂XCF₂CH₃ wherein X is F or Cl, which is a compound useful as a starting material for producing 2,3,3,3-tetrafluoropropene (1234yf), can be obtained by a simple process using relatively inexpensive starting materials.

### DESCRIPTION OF EMBODIMENTS

The present invention will be described below in more detail, with reference to Examples.

### EXAMPLE 1

A 200 mL HASTELLOY autoclave equipped with a stainless-steel gas inlet tube and valve was charged with 19.9 g (92 mmol) of antimony pentafluoride, and sealed. 3.0 g (59 mmol) of methyl chloride and 5.0 g (50 mmol) of tetrafluoroethylene were added thereto in order, and the temperature was raised to 70° C. While maintaining the temperature at 70° C., stirring was conducted for 10 hours. After the autoclave was cooled to room temperature, gas was collected and analyzed by gas chromatography. The results of the analysis are as follows.

CF₃CF₂CH₃: 28% (product selectivity 42%)
CF₂ClCF₂CH₃: 1% (product selectivity 1%)
CF₃CF₂Cl: 14% (product selectivity 21%)

### CH₃F : Undetected

Starting material CF₂=CF₂: 5%
Starting material CH₃Cl: 28%

### Others: 24%

### EXAMPLE 2

The same equipment used in Example 1 was charged with 6.6 g (30 mmol) of antimony pentafluoride, and sealed. 4.3 g (85 mmol) of methyl chloride and 5.3 g (53 mmol) of tetrafluoroethylene were added thereto in order, and the temperature was raised to 70° C. While maintaining the temperature at 70° C., stirring was conducted for 10 hours. After the autoclave was cooled to room temperature, gas was collected and analyzed by gas chromatography. The results of the analysis are as follows.

CF₃CF₂CH₃: 10% (product selectivity 63%)
CF₂ClCF₂CH₃: 1% (product selectivity 6%)
CF₃CF₂Cl: 4% (product selectivity 25%)

### CH₃F: Undetected

Starting material CF₂=CF₂: 38%
Starting material CH₃Cl: 46%

### Others: 1%

### EXAMPLE 3

In a similar manner to Example 1, a 100 mL corrosion resistant autoclave equipped with a stainless-steel gas inlet tube and valve was charged with 8.6 g (28.8 mmol) of antimony pentachloride, and sealed. 3.0 g (60 mmol) of methyl chloride, and 3.0 g (30 mmol) of tetrafluoroethylene were added thereto in order. Stirring was then conducted for 5 hours at 110° C., and for 8 hours at 135° C. After the autoclave was cooled to room temperature, gas was collected and analyzed by gas chromatography. The results of the analysis are as follows. Of the resulting products, CF₃CF=CH₂ was regarded as derived from CF₃CF₂CH₃.

CF₃CF=CH₂: 1% (product selectivity 2%)
CF₃CF₂CH₃: 4% (product selectivity 8%)
CF₃CF₂Cl: trace
CF₂ClCF₂Cl: 44% (product selectivity 88%)
CH₂Cl₂: 1% (product selectivity 2%)

### EXAMPLE 4

The antimony halide used in Example 1 was taken out under a dry atmosphere, and then added into another 200 mL HASTELLOY autoclave. After sealing the autoclave, 50 g of hydrogen fluoride was added thereto, and heating was conducted at 60° C. for 10 hours. Reactivation treatment was thus conducted.

After the autoclave was cooled to room temperature, hydrogen fluoride in the autoclave was collected under reduced pressure. The same amounts of methyl chloride and tetrafluoroethylene as in Example 1 were added to the autoclave, and allowed to react at 70° C. for 10 hours as in Example 1. After the autoclave was allowed to cool to room temperature, gas was collected and analyzed by gas chromatography. The analysis revealed that CF₃CF₂CH₃ and CF₂ClCF₂CH₃, which were derived from a coupling product of tetrafluoroethylene and methyl chloride, were synthesized as in Example 1.

CF₃CF₂CH₃: 8% (production selectivity 62%)
CF₂ClCF₂CH₃: 1% (production selectivity 8%)
CF₃CF₂Cl: 3% (production selectivity 23%)

### CH₃F: Undetected

Starting material CF₂=CF₂: 40%
Starting material CH₃Cl: 47%
Others: 1%

1. A process for preparing a fluorine-containing propane represented by the formula: CF₂XCF₂CH₃ wherein X is F or Cl, the process comprising reacting tetrafluoroethylene and methyl chloride in the presence of an antimony halide represented by the formula: SbFₓCl₅₋ₓ wherein x is a value of 0 to 5.

2. The process according to claim 1, wherein the antimony halide is antimony pentafluoride.

3. The process according to claim 2, wherein the antimony pentafluoride is obtained by bringing an antimony halide represented by the formula: SbFₓCl₅₋ₓ, wherein x is a value of not less than 0 and less than 5, into contact with a fluorinating agent to convert the antimony halide into the antimony pentafluoride.

4. The process according to claim 1, wherein the antimony halide is used in an amount of 0.01 to 3 mol per mol of tetrafluoroethylene.

5. The process for preparing a fluorine-containing propane according to claim 1, wherein the antimony halide is prepared by bringing the antimony halide, which has been used in the production of the fluorine-containing propane by the process of claim 1, into contact with an oxidizing agent and/or a fluorine-containing compound to thereby reactivate the antimony halide.

6. The process according to claim 5, wherein the oxidizing agent is chlorine, and the fluorine-containing compound is hydrogen fluoride.
